(19) United States
(12) Patent Application Publication
(10) Pub. No.: US 2014/0031658 A1
(43) Pub. Date: Jan. 30, 2014

(54) SYSTEM AND METHOD FOR MONITORING GLUCOSE OR OTHER COMPOSITIONS IN AN INDIVIDUAL

(71) Applicants: Hui Zheng, Brookline, MA (US); David A. Schoenfeld, Sharon, MA (US); David M. Nathan, Newton, MA (US)

(72) Inventors: Hui Zheng, Brookline, MA (US); David A. Schoenfeld, Sharon, MA (US); David M. Nathan, Newton, MA (US)

(21) Appl. No.: 14/110,801

(22) PCT Filed: Apr. 11, 2012
(86) PCT No.: PCT/US12/32992

§ 371 Date: Oct. 9, 2013

Related U.S. Application Data
(60) Provisional application No. 61/474,454, filed on Apr. 12, 2011.
(60) Provisional application No. 61/474,439, filed on Apr. 12, 2011.

(51) Int. Cl. A61B 5/145

(52) U.S. Cl. CPC A61B 5/14532
USPC 600/365

(57) ABSTRACT

A system and method for modeling a blood glucose (BG) level of an individual is presented. A continuous glucose monitor (CGM) device is configured to monitor a blood glucose level of the individual. A processor is configured to receive CGM data of the individual from the CGM device, smooth the CGM data into a plurality of continuous curves, and generate an individual-level model of a BG profile of the individual using the plurality of continuous curves. The processor is configured to estimate the average blood glucose curve and inter-day variance-covariance of BG within the individual using the individual-level model, and generate a report based on the average blood glucose curve and inter-day variance-covariance of BG within the individual.

### CROSS-REFERENCE TO RELATED APPLICATIONS

This application is based on and claims the benefit of U.S. Provisional Patent Application Ser. No. 61/474,439, filed Apr. 12, 2011, and entitled “METHODS AND SYSTEMS FOR MONITORING GLUCOSE AND OTHER COMPOSITIONS IN A PATIENT,” which is hereby incorporated by reference.

This application is based on and claims the benefit of U.S. Provisional Patent Application Ser. No. 61/474,454, filed Apr. 12, 2011, and entitled “METHODS AND SYSTEMS FOR MONITORING GLUCOSE AND OTHER COMPOSITIONS IN A PATIENT,” which is hereby incorporated by reference.

### STATEMENT REGARDING FEDERALLY SPONSORED RESEARCH OR DEVELOPMENT

Not applicable.

### FIELD OF USE

The subject matter disclosed herein relates generally to monitoring systems and methods, and, more particularly, to a system and method for monitoring glucose and other compositions within a patient or individual.

### BACKGROUND OF THE INVENTION

Continuous glucose monitoring (CGM) devices provide frequent measurements, for example, every 1-5 minutes, of interstitial glucose levels. As such, these devices provide a powerful tool for both treating patients and studying blood glucose (BG) control strategies. Unlike older techniques that involve only infrequent blood glucose (BG) sampling, which can provide unrepresentative and misleading results, CGM provides more frequent measurements throughout the day, allowing a more comprehensive and detailed assessment of a patient's glucose trend.

With these improved capabilities, CGM systems have been shown to be useful in improving BG control in selected diabetic patient populations. But the clinical role of these devices is still being determined. The best ways for clinicians and patients to utilize the mass of data, taking into account the frequent intra- and inter-day fluctuations and measurement error, remains unclear.

For example, CGM data can be seen to have multiple levels of variation within or between various time periods, thereby making it difficult to develop indicators or predictive metrics of a patient's BG throughout a given time period. As a first example, many days of CGM data from a patient often demonstrate a substantial degree of inter-day variability. For illustrations, FIGS. 1A-1H are graphs showing the CGM BG variance in a single patient fitted over 1, 2, 29, 30, 57, 58, 87, and 88 days, respectively. The assessment of a 24 hour curve representing the patient's typical daily glucose pattern could provide the basis for developing a BG control strategy. This would require summarizing measurements taken over multiple days into a single representative trend. It is also important to estimate the expected amount of inter-day variation to detect any significant deviation from the target range. As a second example, CGM data also presents varying patterns from patient to patient. To identify outliers in clinical practice, methodologies are needed to estimate a group-wide “average profile” and the amount of inter-patient variation. Finally, measurement errors must be factored into the assessment of self-monitoring clinical data.

Therefore, it would be advantageous to have a system and method for analysis of glucose levels measured with CGM devices. Also, it would be advantageous to have systems and methods that are capable of assisting clinicians and patients process CGM data, providing insights into the patterns of glycemia and the treatments necessary to control glucose using CGM data, and providing a useful analytic tool in research and other studies.

### BRIEF DESCRIPTION OF THE INVENTION

The present invention overcomes the aforementioned drawbacks by providing a system and method for analyzing and providing useful reports based on continuous glucose monitoring (CGM) data. The method starts by taking a plurality of functions (curves) that are then used to generate an individual-level model of a BG profile of the individual. An estimate of inter-day variance-covariance of BG within the individual can be created using the individual-level model. Accordingly, the wealth of data associated with CGM devices can be process and useful metrics and reports, for both clinical and research purposes, can be provided.

In one implementation, the present invention is a system for modeling a blood glucose (BG) level of an individual. The system includes a continuous glucose monitor (CGM) device configured to monitor a blood glucose level of the individual, and a processor. The processor is configured to receive CGM data of the individual from the CGM device, smooth the CGM data into a linear combination of a plurality of continuous curves, and generate an individual-level model of a BG profile of the individual using the plurality of continuous curves. The processor is configured to estimate inter-day variance-covariance of BG within the individual using the individual-level model, and generate a report based on the inter-day variance-covariance of BG within the individual.

In another implementation, the present invention is a method for modeling a blood glucose (BG) level of an individual. The method includes capturing continuous glucose monitoring (CGM) data of the individual, smoothing the CGM data into a plurality of spline curves, and generating an individual-level model of a BG profile of the individual using the plurality of spline curves. The method includes estimating inter-day variance-covariance of BG within the individual using the individual-level model, and generating a report based on the step of estimating.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

The present invention will become more apparent from the detailed description set forth below when taken in conjunction with the drawings, in which like elements bear like reference numerals.

FIGS. 1A-1H are graphs showing the CGM BG in a single patient on days 1, 2, 29, 30, 57, 58, 87, and 88, respectively.

FIG. 2 is a diagram showing a system for continuous monitoring of patient's BG data and for implementing the present method

FIG. 3 is a flowchart illustrating an example method for analyzing an individual's (or a group's) CGM data.

FIG. 4 is a graph showing the three basis functions B₃(t),B₄(t) and B₅(t).

FIG. 5A is a graph showing a covariance function of a daily glucose curve for an individual.

FIG. 5B is a graph showing covariance function for the individual average curve.

FIG. 6 is a graph showing the smoothed intra-day blood glucose curve and its 95% credible interval overlapped with the measured blood glucose of a type 1 diabetes patient over a 24-hour period.

FIGS. 7A and 7B shows the group-wide BG curve and 95% credible (confidence) interval of type 1 diabetes patients treated with insulin pump and multiple daily injections, respectively.

FIG. 8 illustrates the estimated difference between MDI-treated and pump-treated type 1 diabetes patients' glucose profiles and the 95% credible (confidence) interval of the differences.

FIG. 9 illustrates probabilities for a type 1 diabetes patient to develop hypoglycemia (BG<50 mg/dl) and hyperglycemia (BG>160 mg/dl) over a time period.

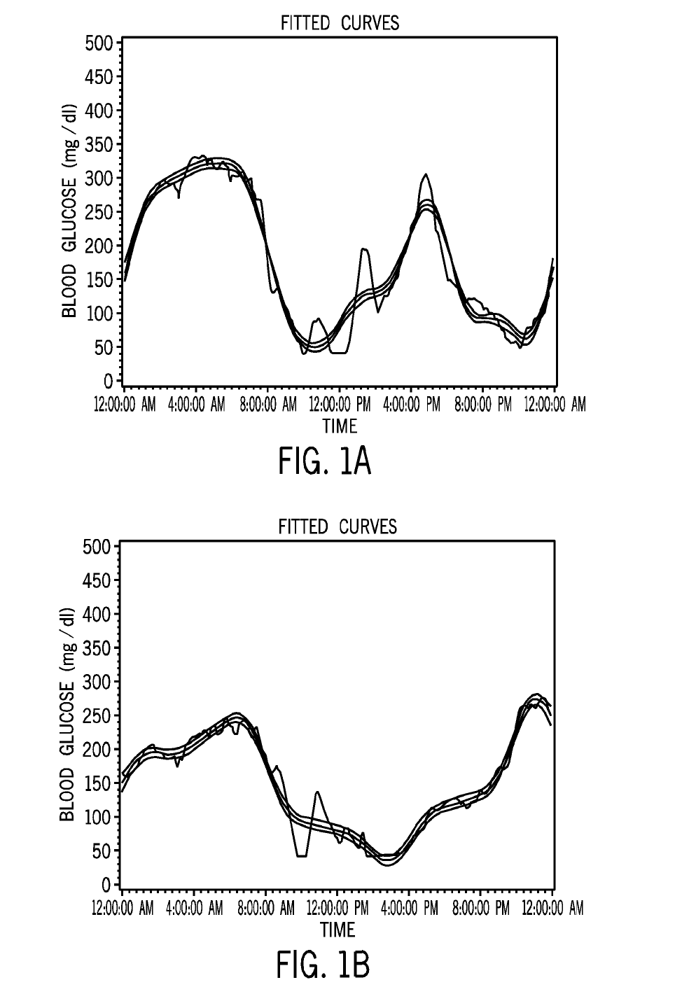

### DETAILED DESCRIPTION OF THE INVENTION

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures. The figures depict selected embodiments and are not intended to limit the scope of embodiments of the invention. Skilled artisans will recognize the examples provided herein have many useful alternatives and fall within the scope of embodiments of the invention.

The following description refers to elements or features being “connected” or “coupled” together. As used herein, unless expressly stated otherwise, “connected” means that one element/feature is directly or indirectly connected to another element/feature, and not necessarily mechanically. Likewise, unless expressly stated otherwise, “coupled” means that one element/feature is directly or indirectly coupled to another element/feature, and not necessarily mechanically, such as when elements or features are embodied in program code. Thus, although the figures depict example arrangements of processing elements, additional intervening elements, devices, features, components, or code may be present in an actual embodiment.

The invention may be described herein in terms of functional and/or logical block components and various processing steps. It should be appreciated that such block components may be realized by any number of hardware, software, and/or firmware components configured to perform the specified functions. For example, an embodiment may employ various integrated circuit components, e.g., memory elements, digital signal processing elements, logic elements, diodes, look-up tables, etc., which may carry out a variety of functions under the control of one or more microprocessors or other control devices. Other embodiments may employ program code, or code in combination with other circuit components.

In accordance with the practices of persons skilled in the art of computer programming, the present disclosure may be described herein with reference to symbolic representations of operations that may be performed by various computing components, modules, or devices. Such operations may be referred to as being computer-executed, computerized, software-implemented, or computer-implemented. It will be appreciated that operations that can be symbolically represented include the manipulation by the various microprocessor devices of electrical signals representing data bits at memory locations in the system memory, as well as other processing of signals. The memory locations where data bits are maintained are physical locations that have particular electrical, magnetic, optical, or organic properties corresponding to the data bits.

The various aspects of the invention will be described in connection with monitoring systems and methods, and, more particularly, to a system and method for monitoring glucose and other compositions within a patient or an individual. However, it should be appreciated that the invention is applicable to other procedures and to achieve other objectives as well.

The present system and method, in one aspect, creates a model for CGM BG data using a multi-level random effects model. The approach focuses on estimating individual-level and group-level profiles and their respective variability using a statistical methodology for comparison of profiles between groups. This model smoothes CGM data into continuous curves, and can be used to estimate an individual patient's glucose profiles, estimate the probabilities for the patient(s) to develop hyperglycemia or hypoglycemia, estimate group-wide glucose profiles, estimate inter-day variance-covariance within a patient, estimate inter-individual variance-covariance, and draw statistical comparisons among groups of patients who differ in characteristics such as disease type, treatment method, genetic trait, and the like. This is in contrast to existing modeling systems that are only arranged to perform rudimentary analysis of BG data. Some systems, for example, only estimate a BG profile as a step function that is constant throughout a time period (e.g., an hour). These systems fail to incorporate correlations between neighboring time points and cannot incorporate prior information.

In one implementation, the system first uses a set of independent nonlinear continuous functions (e.g., B-spline functions) defined in a 0-24 hour time interval to smooth raw BG data (collected, for example, from a CGM device). Then a number of parameters can be used to describe the BG curve for each day. A summary curve can then be generated. The summary curve is the continuous (e.g., spline) function with coefficients that are the mean of the coefficients of daily curves. This approach allows for the smoothing of neighboring data points first, followed by an averaging across a number of days. While generating the summary curve, correlations among BG are estimated for any time points in a typical 24 hour period. Those estimates then allow for the estimation of a prediction interval of the BG curve and the estimation of probabilities for the patient(s) to develop hyperglycemia or hypoglycemia. The use of B-splines in the present disclosure represents one suitable implementation of the present system and method. Other sets of independent curves defined on the interval from 0 to 24 hours could be used for this purpose although some sets of curves would require more or less curves.

Using the present system, therefore, certain health risks, such as hyperglycemia and hypoglycemia can be avoided. By analyzing the BG predictions generated by the system, the probability of going above or below user-defined thresholds for BG levels (e.g., a maximum of 120 mg/dL or a minimum of 60 mg/dL) can be calculated. Those probabilities can then be used by a doctor to better understand a patient's health risks and to guide the patient's treatment.

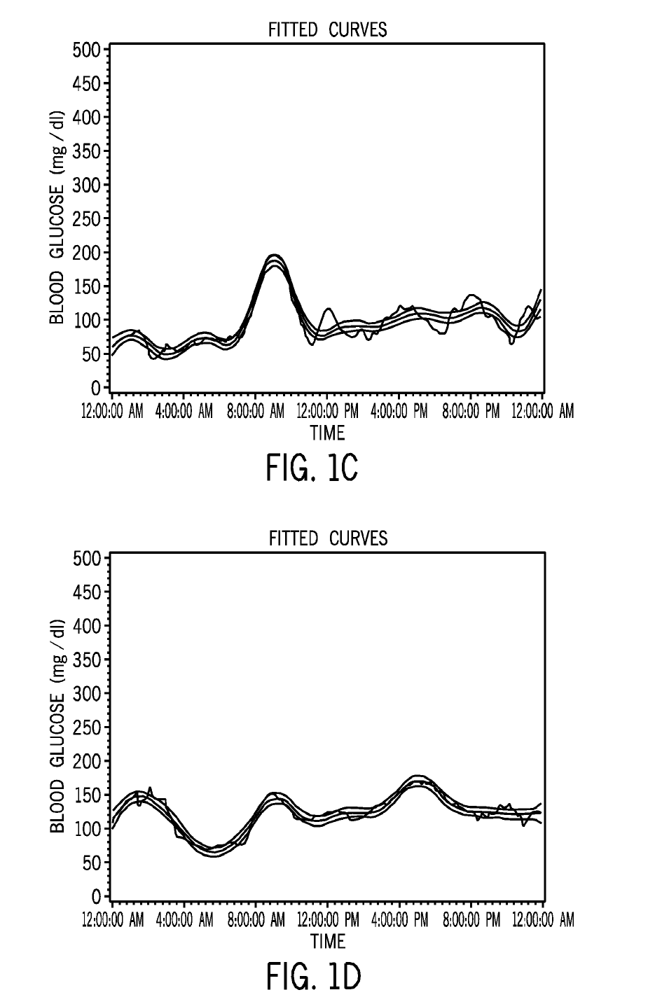

FIG. 2 is an illustration showing a system for continuous monitoring of patient's BG data and for implementing the present method. Monitoring system 10 includes a CGM device 12. CGM device 12 is in communication with blood glucose meter 18 (or other portable device, such as a mobile phone, tablet computer, or other portable device) and is configured to make routine measurements of a user's BG. For example, CGM device 12 may take a measurement every 5 minutes, for a total of 288 measurements per day. Data captured by CGM device 12 may be communicated to blood glucose meter 18 (or another device) for analysis. Blood glucose meter 18 and/or CGM device 12 may optionally be in communication with alarm and injection apparatus 14 for administering a treatment to a user or alerting a user to a dangerous condition.

Communication between the various devices of the monitoring system may be accomplished through any suitable wired and/or wireless connection, such as infrared, Bluetooth, wireless local area network, local area network, and the like. In some embodiments, a relay transmitter may be provided for receiving signals indicative of blood glucose level from blood glucose meter 18. The relay transmitter may receive signals from any of the CGM device 12, the alarm and injector apparatus 14 and/or the blood glucose meter 18. The signals may be transmitted from the relay transmitter (e.g., a bedside relay transmitter) to a remote receiver, which may include an alarm, for example, for alerting a caregiver of the low blood glucose condition.

The CGM device 12 can be used to obtain time-resolved BG data from the user. That data can then be transmitted to blood glucose meter 18 where it is analyzed in accordance with the present method to generate a model by which the user's BG levels may be predicted.

While CGM device 12 may communicate such data to blood glucose meter 18 for processing, CGM device 12 may, itself, include a processor for performing analysis of the captured data. Additionally, the data captured by CGM device 12 may be communicated to devices other than blood glucose meter 18, such as a handheld computing device, cellular phone, or other computing device for processing and analysis.

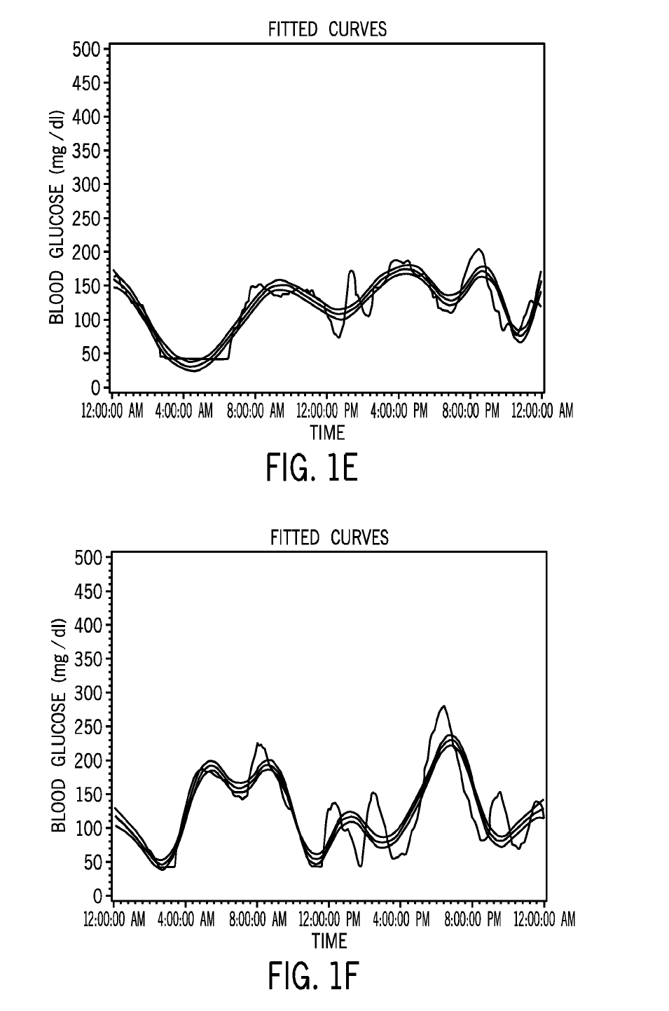

FIG. 3 is a flowchart illustrating an example method for analyzing an individual's (or a group's) CGM data. The method can be used to analyze any CGM data, however the following descriptions presumes the availability of interstitial glucose level measurements taken at a particular duration, for example, every 5 minutes, resulting in a set of values, for example, 288 values, recorded each day for a patient. The following analysis can be performed, for example, on such data collected for a large number of individual patients, however the present system and method may alternatively be utilized to analyze datasets comprising a different number of values with different sample frequency.

A glucose curve can be modeled as the linear combination of a set of independent non-linear continuous functions, for example M (e.g.,14) quadratic B-spline basis functions. Quadratic B-splines are piece-wise quadratic functions that are continuous to the first derivative and have discontinuities in the second derivative at some points called knots. These basis functions can be derived recursively using the De Boor algorithm (see, for example, De Boor C: Package for calculating with B-splines. SIAM J Numerical Anal 1977; 14:441-472, and De Boor C: A Practical Guide to Splines. Berlin: Springer, 1978.).

The 0^{th} degree B-spline basis functions are [symbol] _{i,0}(t)=1 if κᵢ≦t≦κᵢ₊₁, and [symbol] _{i,0}(t)=0 if t<κᵢ or t>κᵢ₊₁.

The d-th degree basis functions are obtained by the recursive relation

$${B}_{i , d} ( t ) = \frac{( t - {κ}_{i} )}{( {κ}_{i + d} - {κ}_{i} )} {B}_{i , d - 1} ( t ) + \frac{( {κ}_{i + d + 1} - t )}{( {κ}_{i + d + 1} - {κ}_{i + 1} )} {B}_{i + 1 , d - 1} ( t ) .$$

Alternatively, the basis functions can be derived using the following explicit form. Based on 17 knots positioned at κᵢ=−4, −2, 0, . . . , 24, 26, 28, the 14 quadratic B-spline basis functions take the following form:

$${B}_{i , 2} ( t ) = \{ \begin{matrix}0 & if t > {κ}_{i + 3} or t < {κ}_{i} \\ \frac{{( t - {κ}_{i} )}^{2}}{8} & if {κ}_{i} \leq t < {κ}_{i + 1} \\ \frac{( {κ}_{i + 2} - t ) ( t - {κ}_{i} ) + ( t - {κ}_{i + 1} ) ( {κ}_{i + 3} - t )}{8} & if {κ}_{i + 1} \leq t < {κ}_{i + 2} \\ \frac{{( {κ}_{i + 3} - t )}^{2}}{8} & if {κ}_{i + 2} \leq t \leq {κ}_{i + 3}\end{matrix}$$

Here knots −4, −2, 26 and 28 are added to facilitate the calculation of basis functions in the end intervals (0-4) hours and (20-24) hours. They are only needed for mathematical convenience and do not bear physical interpretation.

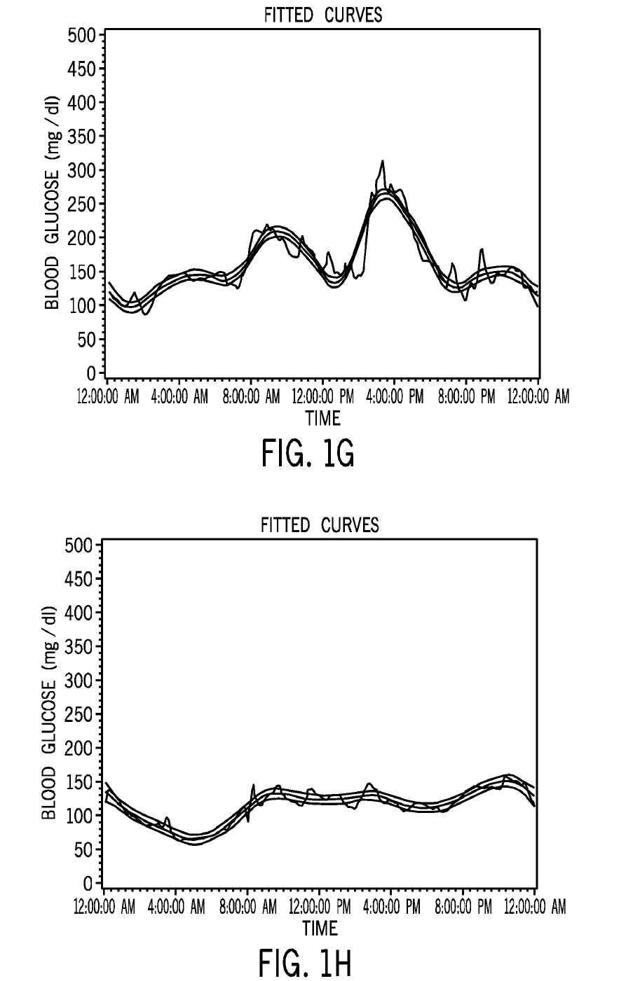

FIG. 4 is a graph showing the three basis functions [symbol] ₃(t), [symbol] ₄(t) and [symbol] ₅(t).

A three-level quadratic B-spline model can be used to model the group-wide, individual-level and intra-day blood glucose variation or variance-covariance.

Referring back to FIG. 3, in step 100, a first model is generated for an individual j's BG level for each hour during a day k. Individual j's BG level at time t∈(0-24) hours on day [symbol] is assumed to be [symbol] (t)= [symbol] (t)+ [symbol] (t), where [symbol] (t) is a 2^{nd} degrees (quadratic) spline function, such as:

$${f}_{jk} ( t ) = {\sum }_{l = 1}^{M} {a}_{ljk} {B}_{l} ( t ) . \qquad Equation ( 1 )$$

In this example, M is assigned a value of 14. Equation (1) has basis functions [symbol] (t), [symbol] =1, . . . , 14 and parameters ( [symbol] , . . . , [symbol] ). The 14 quadratic (d=2) basis functions can be based on 17 evenly spaced knots at −4, −2, 0, 2, . . . , 24, 26, 28 hours. The knots at −4, −2 and 26, and 28 hours can be added to aid the computation of basis functions in the 0-4 and 20-24 hour intervals, but do not bear any physical interpretation. The errors ( [symbol] (t)) are assumed to be independent and identically distributed (i.i.d.) normally distributed with mean zero and variance σ².

In step 102 an individual-level profile is generated for individual j's daily BG about a mean trend function. Here, it is assumed that the daily spline coefficients of individual j were normally distributed around the individual mean with a common covariance matrix, as follows:

( [symbol] , . . . , [symbol] )˜Normal((a₁ⱼ, . . . , a₁₄ⱼ),Φ) Equation (2).

Using this structure, the individual mean trend function (i.e., an individual-level profile) is defined as

$${f}_{j} ( t ) = {\sum }_{l = 1}^{14} {a}_{lj} {B}_{l} ( t ) .$$

It may be assumed that all within-individual, day to day covariance matrices are the same (Φ). If necessary and computationally feasible, this assumption can be relaxed to allow the variance matrix to vary with individual.

In step 104, for an individual j, the inter-day variation of blood glucose function at time point t can be modeled using individual j's BG level for each hour during a day k (determined in step 100). That model is given by:

$$Var ( {f}_{jk} ( t ) \vert j ) = Var ( {\sum }_{l = 1}^{14} {a}_{ljk} {B}_{l} ( t ) ) = {B ( t )}^{T} Φ B ( t ) \qquad Equation ( 3 )$$

In step 106, a model is generated for the group-level profile using the individual-level profile generated earlier. Here, it is assumed that the individual-specific parameters were normally distributed around a group-wide mean with covariance matrix Σ, as follows:

(a₁ⱼ, . . . , a₁₄ⱼ)˜Normal((a₁, . . . , a₁₄),Σ) Equation (4).

This structure assumes that individual-specific trend functions vary around a group-wide glucose trend function (i.e., a group wide profile)

$$f ( t ) = {\sum }_{l = 1}^{14} {a}_{l} {B}_{l} ( t ) . \qquad$$

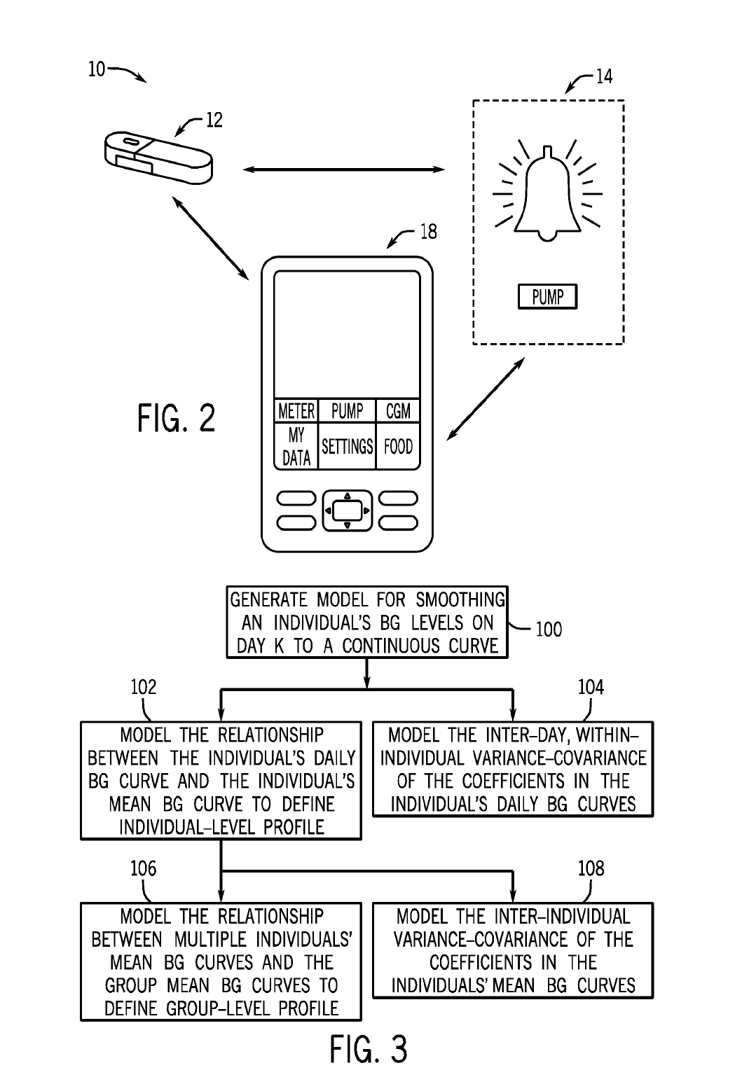

At a typical time point, three consecutive quadratic B-spline basis functions take non-zero values, suggesting that the adjacent parameters are correlated with cov( [symbol] , [symbol] )≠0 for any | [symbol] ₁− [symbol] ₂|≦2. Numerical comparisons show that, for CGM data, unstructured and banded Toeplitz within-individual inter-day Φ matrices lead to very similar estimations for individual and group-wide profiles. FIG. 5A is a graph showing this banded covariance structure. An unstructured covariance matrix was revealed for individual average curve, as shown in FIG. 5B. A Toeplitz structure can be used as the within-individual inter-day covariance Φ when resource limitations such as computer memory and CPU time usage exist.

The model can be viewed as a hierarchical Bayesian model with normal priors for the B-spline coefficients. Bayesian algorithms can be used to draw inferences on individual-level and group-wide profiles.

This model can be parameterized as a three-level linear mixed effects model and fitted using conventional statistical software such as SAS version 9.2 (SAS Institute Inc, Cary, N.C.). In SAS PROC MIXED, this model requires two ‘random’ statements to specify the individual-level and within-individual, day-to-day random effects. Some sample SAS code is given by the following table:

TABLE 1
/*
SAS PROGRAM FOR A THREE-LEVEL B-SPLINE MODEL
DATA1 CONTAINS VARIABLES ID, PLASMABG, DAY, AND
B-SPLINE FUNCTIONS B1-B14.
THE SMOOTHED GLUCOSE CURVES AND 95% CI ARE OUTPUT
INTO DATASET OUTP1.
THE POPULATION AVERAGE CURVE AND 95% CI ARE OUTPUT
IN DATASET OUTPM1.
*/
PROC MIXED DATA=DATA1 METHOD=MIVQUE0;
CLASS ID DAY;
MODEL PLASMABG= B1- B14 /NOINT SOLUTION
DDFM=BETWITHIN OUTP=OUTP1
OUTPM=OUTPM1;
RANDOM B1- B14 / SUBJECT= ID G TYPE=UN;
RANDOM B1- B14 / SUBJECT= DAY(ID) G TYPE=TOEP(3);
RUN;

Using the hierarchical model, it is possible to define and estimate the inter-individual and inter-day variance-covariance matrices of model coefficients and calculate the point-wise variance of the mean glucose functions.

Accordingly, returning to FIG. 3, in step 108, the inter-individual variance of blood glucose function at time point t can be estimated using the following equation:

$$Var ( {f}_{j} ( t ) ) = Var ( {\sum }_{l = 1}^{14} {a}_{lj} {B}_{l} ( t ) ) = {B ( t )}^{T} \sum B ( t ) . \qquad Equation ( 5 )$$

In Equation (5), B(t) is the 14×1 matrix containing the values [symbol] ₁(t), . . . , [symbol] ₁₄(t).

The two quantities in equations (3) and (5) were estimated by replacing Σ and Φ by their maximum likelihood (ML) or restricted maximum likelihood (REML) estimates, \hat{Σ} and \hat{Φ}, respectively.

The standard error (SE) of the group-wide function and individual-specific mean function can be estimated by replacing the variance-covariance matrices in the above equations by the estimated variance-covariance matrices of (â₁ⱼ, . . . , â₁₄ⱼ) and ( [symbol] , . . . , [symbol] ), respectively.

The matrices \hat{Σ} and \hat{Φ}, the variance-covariance matrices of (â₁ⱼ, . . . , â₁₄ⱼ) and ( [symbol] , . . . , [symbol] ), and the error variance σ² can all be estimated under the mixed effects model theory.

This model-based variance estimation requires relatively accurate estimation of the variance-covariance matrices. A robust, albeit computationally intensive, alternative is the replication-based estimation method, which does not involve the estimation of a covariance matrix. A replication-based method can be used to verify the model-based estimation.

For example, the following boostrap method can be used to estimate the standard error of group-wide profiles. 10 replication samples were created by drawing with replacement from the sample the same number of individuals. The three-level model described above is applied to each replication sample to obtain a replicate estimate of the mean glucose trend function. From the replicate estimates, the bootstrap estimate of the point-wise standard error of the mean trend function is calculated. To check the validity of the model-based standard error, the bootstrap variance estimation is compared with the model-based estimation on the type 1 diabetes patients' data from the ADAG study.

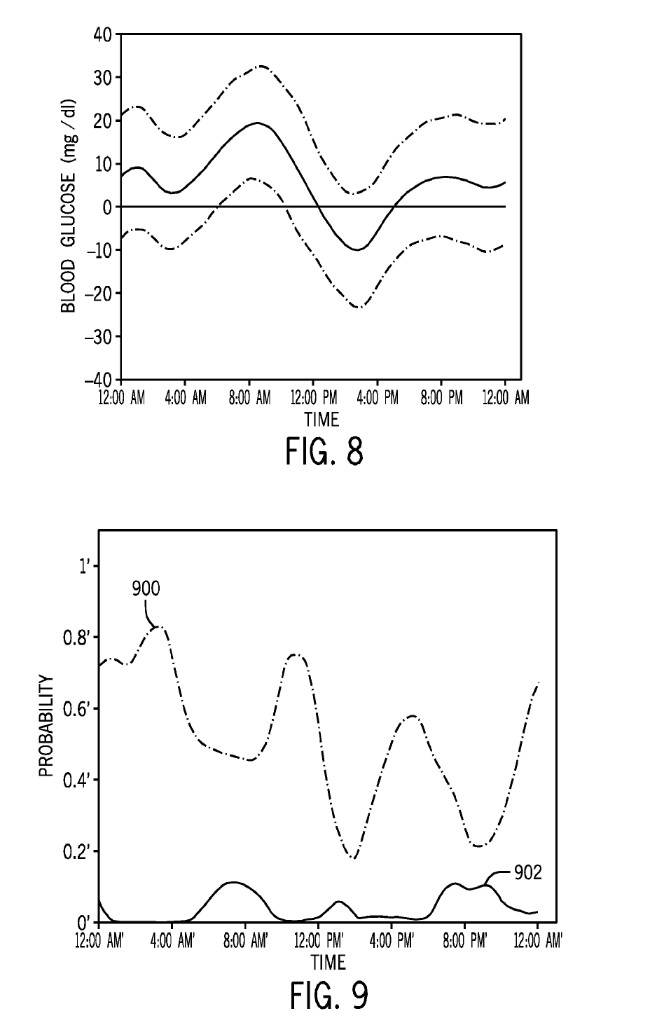

This model, based on the estimated distribution of patient blood glucose, can help estimate the risk for the patient to develop hyperglycemia or hypoglycemia. The risk can be quantified as the probability for the patient's blood glucose value to cross above a given threshold T_{high} (e.g., 160 mg/dL) or drop below a threshold T_{low} (e.g., 50 mg/dL) at any given time point of a typical day. For example, FIG. 9 illustrates the estimated risks (probabilities) for a type 1 diabetes patient to develop hypoglycemia and hyperglycemia over a given time period. In FIG. 9, line 900 represents the estimated probability of hyperglycemia (i.e., BG>160 mg/dL) and line 902 represents the estimated probability of hypoglycemia (i.e., BG<50 mg/dL). As seen in FIG. 9, the individual has a risk of hyperglycemia with typical peaks after meal time and a low chance of hypoglycemia.

Conditioned on the within-patient, inter-day variance-covariance matrix for patient j, Φ, σ² and (a₁ⱼ, . . . , a₁₄ⱼ), the blood glucose at time t is normally distributed with mean

$${f}_{j} ( t ) = {\sum }_{i = 1}^{14} {a}_{ij} {B}_{i} ( t )$$

and variance B(t)^{T}ΦⱼB(t)+σ². The probability for a single glucose reading at time t to exceed T_{high} is

$$1 - {F}_{0} ( \frac{( {T}_{high} - {f}_{j} ( t ) )}{\sqrt{{B ( t )}^{T} {Φ}_{j} B ( t ) + {σ}^{2}}} ) ,$$

where F₀ is the cumulative distribution function (cdf) of the standard normal distribution.

Similarly, the probability for a single glucose reading at time t to drop below T_{low} is

$${F}_{0} ( \frac{( {T}_{low} - {f}_{j} ( t ) )}{\sqrt{{B ( t )}^{T} {Φ}_{j} B ( t ) + {σ}^{2}}} ) .$$

The above two probabilities can be estimated by substituting (a₁ⱼ, . . . , a₁₄ⱼ),Φ and σ² by their corresponding estimates in the above formulas, respectively. In a fully Bayesian approach, these two probabilities are estimated from the posterior distribution of BG(t), which can be drawn with the Monte Carlo Markov Chain (MCMC) method.

To demonstrate the utility of the proposed model in comparing blood glucose profiles, an experiment was performed to compare two groups of type 1 diabetes patients: group 1 was insulin pump-treated and group 2 was treated with MDI. The goal of the experiment was to assess how much, on average, the use of an insulin pump influenced the glucose profile in type 1 diabetes. In order to perform this exemplary comparison, a group indicator Iⱼ was defined that had a value of 0 if individual j was in group 1, and 1 if the individual was in group 2.

Fourteen additional parameters (Δ₁, . . . , Δ₁₄) were introduced that represented the differences in spline coefficients between group 2 and group 1. The group model, see Equation (4), was then modified to be (a₁ⱼ, . . . , a₁₄ⱼ)˜Normal((a₁+Δ₁Iⱼ, . . . , a₁₄+Δ₁₄Iⱼ),Σ) so that group 1 and group 2 were modeled to have different profiles. In the mixed effects model, parameters (Δ₁, . . . , Δ₁₄) were treated as fixed effects. One can use the Hotelling T-square test (see Hotelling H. The generalization of Student's ratio. Annals of Mathematical Statistics. 1931; 2(3):360-378.) for the null hypothesis that Δ₁=Δ₂= . . . =Δ₁₄=0. One can use Bayesian models to allow either identical or different covariance matrices in the comparison groups. For pump vs. MDI comparison, the two groups are assumed to have the same covariance matrices, although it is possible to model them to be different.

The difference of the two spline functions is still a spline function with parameters (Δ₁, . . . , Δ₁₄). The estimates (\hat{Δ}₁, . . . , \hat{Δ}₁₄) and their covariance matrix are useful for making a number of comparisons. Point-wise estimates and credible intervals (CI, Bayesian language for confidence interval) of the difference between the two glucose profiles can be obtained at any time point. The difference can be plotted versus time in the form of a spline function (D-curve) and its CI. From the 95% CI of the D-curve it is possible to identify time periods in which two profiles are significantly different.

The areas under the curve (AUC) of two group-wide trend functions can also be compared. This requires the calculation of AUC for each basis function, which equals 1/3 for [symbol] ₁(t) and [symbol] ₁₄(t), 5/3 for [symbol] ₂(t) and [symbol] ₁₃(t), and 2 for [symbol] ₃(t) to [symbol] ₁₂(t).

Comparisons of non-linear quantities such as maximum and minimum differences are possible using simulations. One can draw from the joint posterior distribution of (Δ₁, . . . , Δ₁₄) and obtain distributions of such quantities for statistical inference.

In one experiment, the CGM data from 322 patients with type 1 diabetes, 223 with type 2 diabetes, and 86 non-diabetic subjects was analyzed. Among the type 1 diabetes patients, 124 were treated with an insulin pump and 144 were treated with MDI. The median age of the patients was 45 years (range 16-70) and fifty-three percent were female. The characteristics of the study group are shown in Table 2, below:

TABLE 2

| | Type 1 diabetes | Type 2 diabetes | Non-diabetic |
|---|---|---|---|
| Number | 322 | 223 | 86 |
| Age ± SD (years) | 43 ± 13 | 55 ± 9 | 40 ± 14 |
| Number (%) of | 166 (52%) | 111(50%) | 59 (69%) |
| Females | | | |
| HbA1c ± SD (%) | 7.3 ± 1.2 | 6.8 ± 1.1 | 5.2 ± 0.3 |

Using the three-level spline model, it was possible to obtain patient-specific and group-wide glucose functions for type 1 and type 2 diabetes and for the non-diabetic subjects. It was also possible to estimate the 95% CI for each diabetes type.

Table 3, below, shows the 24 hour average inter-day and inter-patient variations in the three groups: type 1 diabetes, type 2 diabetes and non-diabetic subjects. Type 1 and type 2 diabetes patients had similar inter-patient variability with an average standard deviation of approximately 49 and 43 mg/dl, respectively, compared with 15 mg/dl for the non-diabetic subjects. Type 1 diabetes patients showed higher inter-day variability than type 2 patients (SD=67 vs. 41 mg/dl, p<0.001 according to the estimated asymptotic distribution of the covariance parameters). Type 1 diabetes patients' inter-day variability was higher than the inter-patient variability (SD=67 vs. 49 mg/dl). Type 1 diabetes had the highest residual standard deviation of 19 mg/dl versus 14 mg/dl for type 2 diabetes and 8 mg/dl for non-diabetics.

The model-based standard error estimate was close to the bootstrap estimates for all patient groups. For example, the 24-hour average model-based standard error estimate was 3.1 mg/dl for type 1 diabetes patients, close to the 24-hour average bootstrap estimate of 2.9 mg/dl.

TABLE 3

| | Average | Average | Average |
|---|---|---|---|
| | Inter-patient | Inter-day | Residual |
| | standard | standard | Standard |
| | deviation | deviation | deviation |
| | (mg/dl) | (mg/dl) | (mg/dl) |
| Type 1 diabetes | 49 | 67 | 19 |
| Type 2 diabetes | 43 | 41 | 14 |
| Non-diabetic | 15 | 18 | 8 |

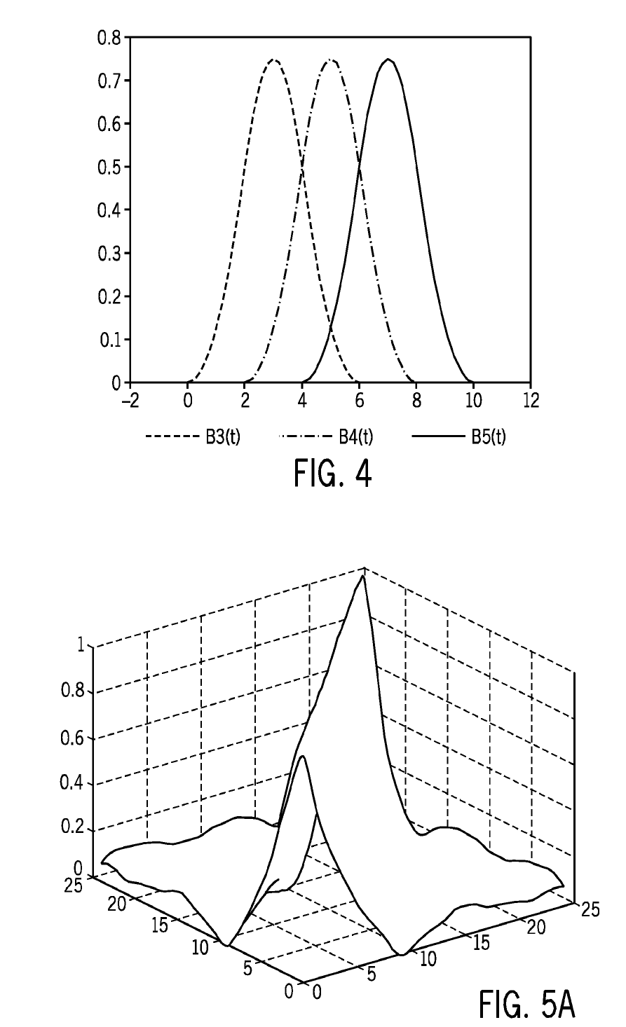

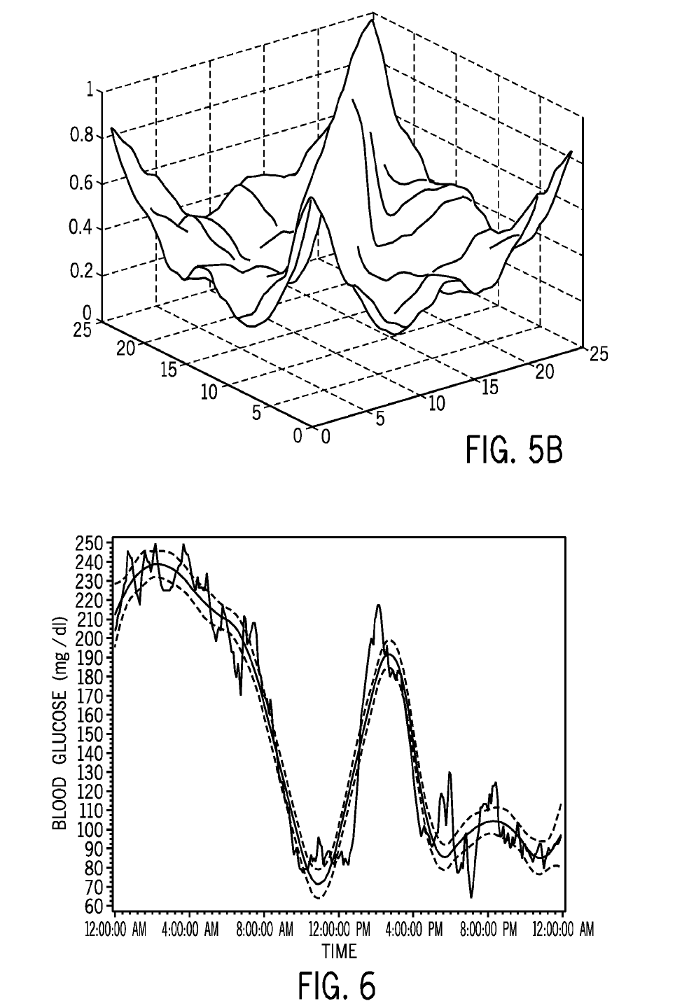

The quadratic B-spline model provides smooth curves tightly tracking an individual patient's CGM measurements. FIG. 6 is a graph showing the smoothed intra-day blood glucose curve and its 95% CI overlapped with the measured blood glucose of a type 1 diabetes patient over a 24-hour period. FIG. 7A is a graph showing the group-wide 24-hour glucose curve for Type 1 diabetic patients treated with an insulin pump. The group-wide glucose curve of such patients trended within the 145-180 mg/dl range and had peaks at three time points: at approximately 9 AM, 3 PM, and midnight, corresponding to the effects of three meals. By comparison, the group-wide 24-hour glucose curves for Type 1 diabetic patients treated with MDI trended within the 145-190 mg/dl range and peaked at approximately 9-10 AM and midnight, as shown by the graph of FIG. 7B.

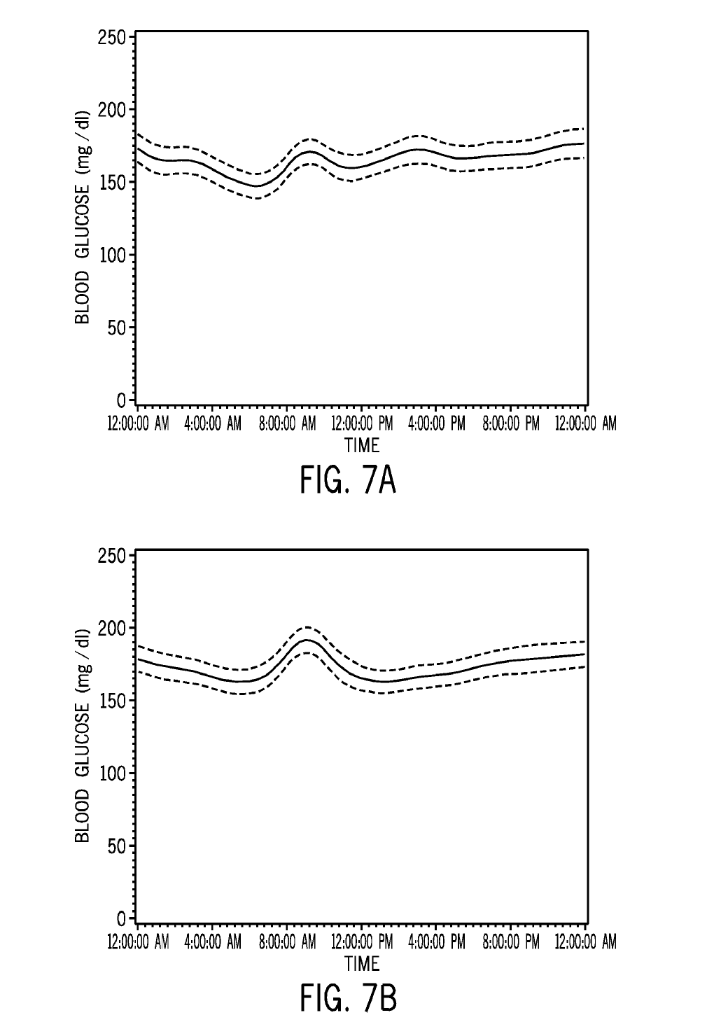

FIG. 8 illustrates the estimated difference between MDI-treated and pump-treated type 1 diabetes patients' glucose profiles and the 95% CI of the differences. FIG. 8 shows the two profiles are significantly different from around 6 AM to around 10 AM. The Hotelling T-square test for the equality of the two group level profiles leads to a p-value of 0.14, suggesting no significant difference between the two group-wide profiles as a whole.

The three-level B-Spline model described above provides a framework for the smoothing and inference of the dense glucose data provided by a CGM. The model allows for estimation of the patient-level and group-level mean glucose profiles and their variability, as well as to make statistical comparisons of profiles among different groups.

Using, for example, 17 equally spaced knots, the model yields smooth curves tightly tracking the CGM measurements. Not surprisingly, the population mean glucose profiles reflect the well-recognized effects of eating on glycemic excursions. The model provides insights into the different patterns of glycemic achieved with therapy in type 1 diabetes and, potentially, the timing and types of interventions necessary to address the glucose excursions.

The evenly spaced knots are a natural fit for the CGM measurements by including a similar number of measurements in each time interval between consecutive knots. The model assumes a quadratic glucose curve within a two hour period. This assumption seems to be adequate for estimating patient-specific or group-wide profiles. Statistical criteria for selection of number and placement of knots have been widely discussed. Such methods include Akaike information criterion (AIC), Bayesian information criterion (BIC), cross-validation, and Bayesian model selection methods such as the reversible jump MCMC method. In practice, the choice of knots should be based on a combination of statistical and practical considerations. In a situation where more detailed examination of glucose curves is necessary, a larger number of knots can be used.

The choice of covariance structures has a limited impact on the estimated shapes of trend functions. A mis-specified covariance structure can still lead to unbiased, albeit not the most efficient, estimates of the glucose curves. However, the covariance structures play an important role in variance estimation and hypothesis testing. Like most smoothing techniques, this model assumes random error independent identically distributed with normal distribution with mean zero and constant variance.

Although the replication-based method provides robust variance estimation, it is computationally resource-demanding, making it infeasible for large numbers of patients or large numbers of measurements per patient. The model-based method is computationally efficient for large datasets without sacrificing the quality of variance estimation.

The proposed hierarchical model allows statistical comparisons of point-wise and overall differences between two (or more) profiles, thus providing a useful statistical tool for clinical trials using glucose profiles as an endpoint. Power calculations for such statistical tests are possible using estimated variance components and the expected effect size.

The B-Spline basis functions demonstrate a numerical advantage in variance estimation over some alternatives such as the truncated polynomial basis functions. B-splines lead to sparse and well-conditioned covariance matrices than the latter. The latter is more prone to produce numerically poor-conditioned covariance matrices that lead to unsatisfactory variance estimation.

This model could be used in software to help patients better control their glucose. The mathematical methods for this may involve using variance covariance matrix as part of a prior distribution for the individual parameters that define a patient's daily profile.

Then the patient's daily profile can be estimated from several weeks (e.g., more than two) of data. This profile, along with a record of insulin injections, food intake, and exercise, can be used to determine behavior changes that might improve the patient's glucose control. Clinical trials may compare such a strategy to other treatment strategies.

The methods proposed herein also help improve estimation of instantaneous insulin values by using the model parameters in a prior distribution for each reading. This would involve building software into the device.

The B-splines based Bayesian modeling techniques provide a promising tool for analyzing CGM data in real-time for clinical decision-making and ultimately for the development of “closed-loop” glucose control systems.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Finally, it is expressly contemplated that any of the processes or steps described herein may be combined, eliminated, or reordered. Accordingly, this description is meant to be taken only by way of example, and not to otherwise limit the scope of this invention.

The present invention has been described in terms of one or more preferred embodiments, and it should be appreciated that many equivalents, alternatives, variations, and modifications, aside from those expressly stated, are possible and within the scope of the invention.

We claim:

1. A system for modeling a blood glucose (BG) level of an individual, comprising:
a continuous glucose monitor (CGM) device configured to monitor a blood glucose level of the individual;
a processor, the processor being configured to:
receive CGM data of the individual from the CGM device,
smooth the CGM data into a plurality of continuous curves,
generate an individual-level model of a BG profile of the individual using the plurality of continuous curves,
estimate inter-day variance-covariance of BG within the individual using the individual-level model, and
generate a report based on the inter-day variance-covariance of BG within the individual.

2. The system of claim 1, wherein the processor is configured to:
generate a group-level model using the plurality of continuous curves; and
estimate a group-wide glucose profile using the group-level model.

3. The system of claim 1, wherein the CGM data of the individual includes data collected over a period of time exceeding three days.

4. The system of claim 1, wherein the continuous curves include B-spline curves.

5. The system of claim 4, wherein the B-spline curves are derived recursively using a De Boor algorithm.

6. The system of claim 1, wherein the processor is configured to use the estimation of inter-day variance-covariance of BG to identify a time to administer a treatment to the individual.

7. The system of claim 6, including an injection apparatus configured to receive an instruction from the processor to administer a treatment.

8. A method for modeling a blood glucose (BG) level of an individual, comprising the steps of:
capturing continuous glucose monitoring (CGM) data of the individual;
smoothing the CGM data into a plurality of spline curves;
generating an individual-level model of a BG profile of the individual using the plurality of spline curves;
estimating inter-day variance-covariance of BG within the individual using the individual-level model; and
generating a report based on the step of estimating.

9. The method of claim 8, including:
generating a group-level model using the plurality of spline curves; and
estimating a group-wide glucose profile using the group-level model.

10. The method of claim 8, including analyzing inter-day variance-covariance of BG within the individual to determine a probability of the BG of the individual exceeding a predetermined maximum or minimum value.

11. The method of claim 10, wherein the maximum value is 110-160 mg/dL.

12. The method of claim 10, wherein the minimum value is 40-70 mg/dL.

13. The method of claim 8, wherein the spline curves include B-spline curves.

14. The method of claim 13, wherein the B-spline curves are derived recursively using a De Boor algorithm.

15. The method of claim 8, including using the estimation of inter-day variance-covariance of BG within the individual to identify a time to administer a treatment to the individual.
